(19) United States
(12) Patent Application Publication
(10) Pub. No.: US 2025/0366948 A1
(43) Pub. Date: Dec. 4, 2025

(54) MEDIA COMMUNICATION ADAPTORS IN A SURGICAL ENVIRONMENT

(71) Applicant: DIGITAL SURGERY LIMITED, London (GB)

(72) Inventors: Petros Giataganas, London (GB); Danail V. Stoyanov, London (GB); Imanol Luengo Muntion, Bilbao (ES); Guathier Camille Louis Gras, London (GB)

(21) Appl. No.: 18/707,695

(22) PCT Filed: Nov. 10, 2021
(86) PCT No.: PCT/GR2021/000067

§ 371 Date: May 6, 2024

(51) Int. Cl. A61B 90/00; G16H 10/60; G16H 20/40; G16H 30/20; G16H 30/40; G16H 40/63

(52) U.S. Cl. CPC A61B 90/361; A61B 90/37; G16H 10/60; G16H 20/40; G16H 30/20; G16H 30/40; G16H 40/63

(57) ABSTRACT

Aspects include incorporating media communication adaptors in a surgical environment. Wireless communication can be established between one or more data adaptors and a central processing hub in a surgical system. The one or more data adaptors can be configured to provide surgical data associated with a surgical procedure. Localized processing of the surgical data can be performed at the one or more data adaptors prior to sending the surgical data to the central processing hub. Localized processing of video associated with the surgical data can be performed at one or more video adaptors prior to sending processed video or an output of the video to the central processing hub. The surgical data and the video can be further processed at the central processing hub.

### BACKGROUND

The present invention relates in general to computing technology and relates more particularly to computing technology for providing real-time video and data feeds through media communication adaptors in a surgical environment.

In medical environments, such as a surgical environment or theater, the use of instruments and systems that generate and/or receive video or other types of data during a medical procedure can result in many communication channels operating substantially in parallel. Some instruments, sensors, cameras, and robotic surgery systems, for example, may have various communication interface options available. For instance, some data/video sources may use coaxial cables, High-Definition Multimedia Interface (HDMI) cables, Ethernet cables, Universal Serial Bus (USB) connectors, optical links, proprietary connections, and other such communication interfaces. Routing various cables and physical connections can be challenging in surgical environments, where surgeons and other medical workers may need to have many options (e.g., freedom of position and movement) for examining a patient and performing procedures. The many connection possibilities can also make it challenging to coherently acquire and use data/video collectively from the various sources.

### SUMMARY

According to an aspect, a computer-implemented method establishes wireless communication between one or more data adaptors and a central processing hub in a surgical system, each of the one or more data adaptors can be configured to provide surgical data associated with a surgical procedure. Localized processing of the surgical data is performed at the one or more data adaptors prior to sending the surgical data to the central processing hub. Wireless communication is established between one or more video adaptors and the central processing hub, where at least one of the one or more video adaptors is configured to provide video associated with the surgical data. Localized processing of the video associated with the surgical data is performed at the one or more of the video adaptors prior to sending the video to the central processing hub. The surgical data and the video are captured and processed at the central processing hub.

In addition to one or more of the features described above or below, or as an alternative, further aspects may include receiving video and/or data through one or more wired connections at the central processing hub, and receiving one or more wireless communications at the central processing hub from one or more wireless-enabled sources.

In addition to one or more of the features described above or below, or as an alternative, further aspects may include where wireless communication between the one or more data adaptors and the central processing hub passes through a hub adaptor configured to synchronize the surgical data from the one or more data adaptors and/or synchronize video received from the one or more video adaptors.

In addition to one or more of the features described above or below, or as an alternative, further aspects may include where wireless communication between the one or more video adaptors and the central processing hub passes through the hub adaptor, and hub adaptor is configured to synchronize the surgical data from the one or more data adaptors with the video from the one or more video adaptors.

In addition to one or more of the features described above or below, or as an alternative, further aspects may include configuring one of the one or more data adaptors or one of the one or more video adaptors to operate as the hub adaptor.

In addition to one or more of the features described above or below, or as an alternative, further aspects may include establishing wireless communication between one or more display adaptors and the central processing hub, and transmitting display data and/or video through one or more wireless connections to the one or more display adaptors for output on one or more display devices coupled to the one or more display adaptors.

In addition to one or more of the features described above or below, or as an alternative, further aspects may include identifying, by one of the display adaptors, a user of one of the display devices, and customizing an aspect of a user interface or information presented in the user interface to display on one of the display devices based on identifying the user.

In addition to one or more of the features described above or below, or as an alternative, further aspects may include where performing localized processing of the surgical data includes performing at least a portion of a surgical machine learning process on at least one of the data adaptors.

In addition to one or more of the features described above or below, or as an alternative, further aspects may include where performing localized processing of the video associated with the surgical data includes modifying one or more aspects of the video based on performing at least a portion of a surgical machine learning process on at least one of the video adaptors.

According to another aspect, a system includes a central processing hub, one or more data adaptors, and one or more video adaptors. The one or more data adaptors are configured to perform localized processing of surgical data associated with a surgical procedure and provide the surgical data to the central processing hub through wireless communication. The one or more video adaptors are configured to perform localized processing of video associated with the surgical data and provide the video to the central processing hub through wireless communication.

In addition to one or more of the features described above or below, or as an alternative, further aspects may include one or more video sources and one or more data sources coupled to the central processing hub through wired connections, and one or more wireless-enabled sources coupled to the central processing hub.

In addition to one or more of the features described above or below, or as an alternative, further aspects may include a hub adaptor coupled by a wireless connection to the central processing hub and through two or more wireless connections to the one or more data adaptors and the one or more one or more video adaptors, where the hub adaptor is configured to pair with the central processing hub, the one or more data adaptors, and the one or more one or more video adaptors using a manual mode, a semi-automatic mode, or an automatic mode.

In addition to one or more of the features described above or below, or as an alternative, further aspects may include where the hub adaptor is configured to perform preprocessing of the surgical data and the video prior to sending the surgical data and the video to the central processing hub.

In addition to one or more of the features described above or below, or as an alternative, further aspects may include one or more display adaptors configured to establish wireless communication with the central processing hub and output display data to one or more display devices.

According to another aspect, a computer program product includes a memory device having computer-executable instructions stored thereon, which when executed by one or more processors cause the one or more processors to perform a method. The method includes receiving surgical data through wireless communication from one or more data adaptors at a central processing hub, where the one or more data adaptors are coupled to one or more data sources in a surgical system. The method also includes receiving video associated with the surgical data through wireless communication from one or more video adaptors at the central processing hub, where the one or more video adaptors are coupled to one or more video sources in the surgical system. The method further includes performing a surgical machine learning process to make one or more phase predictions of a surgical procedure based on the surgical data and the video.

In addition to one or more of the features described above or below, or as an alternative, further aspects may include where one or more of the data adaptors or the video adaptors perform one or more detection processes and provide detection information through wireless communication to the central processing hub to support making the one or more phase predictions.

In addition to one or more of the features described above or below, or as an alternative, further aspects may include where one or more of the data adaptors or the video adaptors perform one or more identification processes and provide identification information through wireless communication to the central processing hub to support making the one or more phase predictions.

In addition to one or more of the features described above or below, or as an alternative, further aspects may include where wireless communication between the one or more data adaptors and the central processing hub is modified upon passing through a hub adaptor configured to synchronize the surgical data from the one or more data adaptors and/or synchronize video received from the one or more video adaptors.

In addition to one or more of the features described above or below, or as an alternative, further aspects may include transmitting display data through one or more wireless connections from the central processing hub to one or more display adaptors for output on one or more display devices coupled to the one or more display adaptors, where the display data is customized at one or more display adaptors based on identifying one or more users of the one or more display devices.

In addition to one or more of the features described above or below, or as an alternative, further aspects may include sending one or more configuration commands through wireless communication to reconfigure operation of at least one of the data adaptors or the video adaptors to become a hub adaptor.

Additional technical features and benefits are realized through the techniques of the present invention. Aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the aspects of the invention are apparent from the following detailed description taken in conjunction with the accompanying drawings in which:

FIG. 1 depicts a computer-assisted surgery system according to one or more aspects;

FIG. 2 depicts a system using multiple communication adaptors for wireless communication in a surgical environment according to one or more aspects;

FIG. 3 depicts a system using a hub adaptor and multiple communication adaptors for wireless communication in a surgical environment according to one or more aspects;

FIG. 4 depicts a system for computer-assisted surgery according to one or more aspects;

FIG. 5 depicts a block diagram of adaptor synchronization in a surgical environment according to one or more aspects;

FIG. 6 depicts a system using machine learning for analyzing video and data according to one or more aspects;

FIG. 7 depicts a block diagram of an adaptor for a surgical environment according to one or more aspects;

FIG. 8 depicts a computer system in accordance with one or more aspects; and

FIG. 9 depicts a flowchart of a method for processing and wireless communication between multiple sources and a central processing hub according to one or more aspects.

The diagrams depicted herein are illustrative. There can be many variations to the diagram or the operations described therein without departing from the spirit of the invention. For instance, the actions can be performed in a differing order, or actions can be added, deleted, or modified. Also, the term “coupled” and variations thereof describe having a communications path between two elements and do not imply a direct connection between the elements with no intervening elements/connections between them. All of these variations are considered a part of the specification.

### DETAILED DESCRIPTION

In exemplary aspects of the technical solutions described herein, a computer-assisted surgical (CAS) system is provided that supports real-time acquisition and use of multiple sources in real-time. The system uses media communication adaptors, also referred to as “dongles”, to provide communication interface conversion for wireless transmission of video and other types of data streams between sources and a central processing hub. The dongles can include small footprint devices with one or more communication interfaces configured to accept wired or optical input/output and communicate wirelessly with the central processing hub and/or a hub adaptor. For example, a video source can be coupled to a video adaptor through a video cable. The video adaptor can perform localized processing of the video and send the video to the central processing hub directly or indirectly through a hub adaptor using wireless communication. Similarly, a data source can be coupled to a data adaptor through a data cable, where the data adaptor performs localized processing of the data and sends the data to the central processing hub directly or indirectly through a hub adaptor using wireless communication.

The video can capture one or more angles of a surgical procedure, which may be captured using an endoscopic camera passed inside a patient adjacent to the location of the surgical procedure to view and record one or more actions performed during the surgical procedure, for example. Other examples of video can include observing a user, such as a surgeon or medical assistant, external to the patient. For instance, video can identify a user interacting with a particular user interface or surgical instrument. The video that is captured can be transmitted and/or recorded in one or more examples. In some examples, portions of the video can be analyzed and annotated post-surgery by the central processing hub or another processing system. Data gathered from various surgical instruments can also be captured for transmission and recording. A technical challenge exists to transmit multiple channels of data and video in real-time as a surgical procedure is performed using a CAS system. Exemplary aspects of technical solutions described herein relate to, among other things, devices, systems, methods, computer-readable media, techniques, and methodologies for providing real-time video and data feeds through multiple adaptors with wireless communication interfaces in a surgical environment.

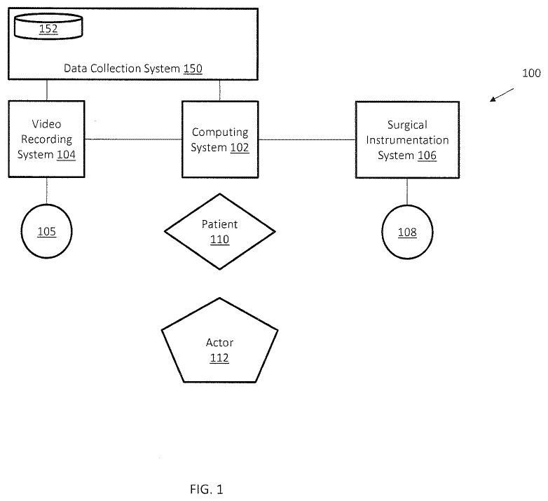

FIG. 1 depicts an example CAS system according to one or more aspects. The CAS system 100 includes at least a computing system 102, a video recording system 104, and a surgical instrumentation system 106. As illustrated in FIG. 1, an actor 112 can be medical personnel that uses the CAS system 100 to perform a surgical procedure on a patient 110. Medical personnel can be a surgeon, assistant, nurse, administrator, or any other actor that interacts with the CAS system 100 in a surgical environment. The surgical procedure can be any type of surgery, such as but not limited to cataract surgery, laparoscopic cholecystectomy, endoscopic endonasal transsphenoidal approach (eTSA) to resection of pituitary adenomas, robotic procedures, or any other surgical procedure. In other examples, actor 112 can be a technician, an administrator, an engineer, or any other such personnel that interacts with the CAS system 100. For example, actor 112 can record data from the CAS system 100, configure/update one or more attributes of the CAS system 100, review past performance of the CAS system 100, repair the CAS system 100, etc.

A surgical procedure can include multiple phases, and each phase can include one or more surgical actions. A “surgical action” can include an incision, a compression, a stapling, a clipping, a suturing, a cauterization, a sealing, or any other such actions performed to complete a phase in the surgical procedure. A “phase” represents a surgical event that is composed of a series of steps (e.g., closure). A “step” refers to the completion of a named surgical objective (e.g., hemostasis). During each step, certain surgical instruments 108 (e.g., forceps) are used to achieve a specific objective by performing one or more surgical actions.

The surgical instrumentation system 106 provides electrical energy to operate one or more surgical instruments 108 to perform the surgical actions. The electrical energy triggers an activation in the surgical instrument 108. The electrical energy can be provided in the form of an electrical current or an electrical voltage. The activation can cause a surgical action to be performed. The surgical instrumentation system 106 can further include electrical energy sensors, electrical impedance sensors, force sensors, bubble and occlusion sensors, and various other types of sensors. The electrical energy sensors can measure and indicate an amount of electrical energy applied to one or more surgical instruments 108 being used for the surgical procedure. The impedance sensors can indicate an amount of impedance measured by the surgical instruments 108, for example, from the tissue being operated upon. The force sensors can indicate an amount of force being applied by the surgical instruments 108. Measurements from various other sensors, such as position sensors, pressure sensors, flow meters, can also be input.

The video recording system 104 includes one or more cameras 105, such as operating room cameras, endoscopic cameras, etc. The cameras 105 capture video data of the surgical procedure being performed. The cameras 105 can capture video in the visible and/or non-visible spectrum, such as infrared, near infrared, ultraviolet, thermal and/or other spectral ranges. The camera may also be another energy source such as a fluoroscopy system which also generate digital video information. The video recording system 104 includes one or more video capture devices that can include cameras 105 placed in the surgical room to capture events surrounding (i.e., outside) the patient being operated upon. The video recording system 104 further includes cameras 105 that are passed inside (e.g., endoscopic cameras) the patient 110 to capture endoscopic data or can be placed outside in exoscope or microscope configurations. The endoscopic data provides video and images of the surgical procedure.

The computing system 102 includes one or more memory devices, one or more processors, a user interface device, among other components. The computing system 102 can execute one or more computer-executable instructions. The execution of the instructions facilitates the computing system 102 to perform one or more methods, including those described herein. The computing system 102 can communicate with other computing systems via a wired and/or a wireless network. In one or more examples, the computing system 102 includes one or more trained machine learning models that can detect and/or predict features of/from the surgical procedure that is being performed or has been performed earlier. Features can include structures such as anatomical structures, surgical instruments 108 in the captured video of the surgical procedure. Features can further include events such as phases, actions in the surgical procedure. Features that are detected can further include the actor 112 and/or patient 110.

Based on the detection, the computing system 102, in one or more examples, can provide recommendations for subsequent actions to be taken by the actor 112. Alternatively, or in addition, the computing system 102 can provide one or more reports based on the detections. The detections by the machine learning models can be performed in an autonomous or semi-autonomous manner.

The machine learning models can include artificial neural networks, such as deep neural networks, convolutional neural networks, recurrent neural networks, encoders, decoders, or any other type of machine learning model. The machine learning models can be trained in a supervised, unsupervised, or hybrid manner. The machine learning models can be trained to perform detection and/or prediction using one or more types of data acquired by the CAS system 100. For example, the machine learning models can use the video data captured via the video recording system 104. Alternatively, or in addition, the machine learning models use the surgical instrumentation data from the surgical instrumentation system 106. In yet other examples, the machine learning models use a combination of video data and surgical instrumentation data.

Additionally, in some examples, the machine learning models can also use audio data captured during the surgical procedure. The audio data can include sounds emitted by the surgical instrumentation system 106 while activating one or more surgical instruments 108. Alternatively, or in addition, the audio data can include voice commands, snippets, or dialog from one or more actors 112. The audio data can further include sounds made by the surgical instruments 108 during their use.

In one or more examples, the machine learning models can detect surgical

actions, surgical phases, anatomical structures, surgical instruments, and various other features from the data associated with a surgical procedure. Features can also be sourced from systems that manage electronic medical records for patients and/or scheduling systems associated with scheduling of surgical procedures. The features can be used to group aspects for machine learning based on various criteria, such as patient characteristics, facilities used, planned procedure duration, and other such parameters. The detection can be performed in real-time in some examples. Alternatively, or in addition, the computing system 102 analyzes the surgical data, i.e., the various types of data captured during the surgical procedure, in an offline manner (e.g., post-surgery). In one or more examples, the machine learning models detect surgical phases based on detecting some of the features such as the anatomical structure, surgical instruments, etc.

A data collection system 150 can be employed to store the surgical data, including the video(s) captured during the surgical procedures. The data collection system 150 includes one or more storage devices 152. The data collection system 150 can be a local storage system, a cloud-based storage system, or a combination thereof. Further, the data collection system 150 can use any type of cloud-based storage architecture, for example, public cloud, private cloud, hybrid cloud, etc. In some examples, the data collection system can use a distributed storage, i.e., the storage devices 152 are located at different geographic locations. The storage devices 152 can include any type of electronic data storage media used for recording machine-readable data, such as semiconductor-based, magnetic-based, optical-based storage media, or a combination thereof. For example, the data storage media can include flash-based solid-state drives (SSDs), magnetic-based hard disk drives, magnetic tape, optical discs, etc.

In one or more examples, the data collection system 150 can be part of the video recording system 104, or vice-versa. In some examples, the data collection system 150, the video recording system 104, and the computing system 102, can communicate with each other via a communication network, which can be wired, wireless, or a combination thereof. The communication between the systems can include the transfer of data (e.g., video data, instrumentation data, etc.), data manipulation commands (e.g., browse, copy, paste, move, delete, create, compress, etc.), data manipulation results, etc. In one or more examples, the computing system 102 can manipulate the data already stored/being stored in the data collection system 150 based on outputs from the one or more machine learning models, e.g., phase detection, structure detection, etc.

Alternatively, or in addition, the computing system 102 can manipulate the data already stored/being stored in the data collection system 150 based on information from the surgical instrumentation system 106.

In one or more examples, the video captured by the video recording system 104 is stored on the'data collection system 150. In some examples, the computing system 102 curates parts of the video data being stored on the data collection system 150. In some examples, the computing system 102 filters the video captured by the video recording system 104 before it is stored on the data collection system 150. Alternatively, or in addition, the computing system 102 filters the video captured by the video recording system 104 after it is stored on the data collection system 150.

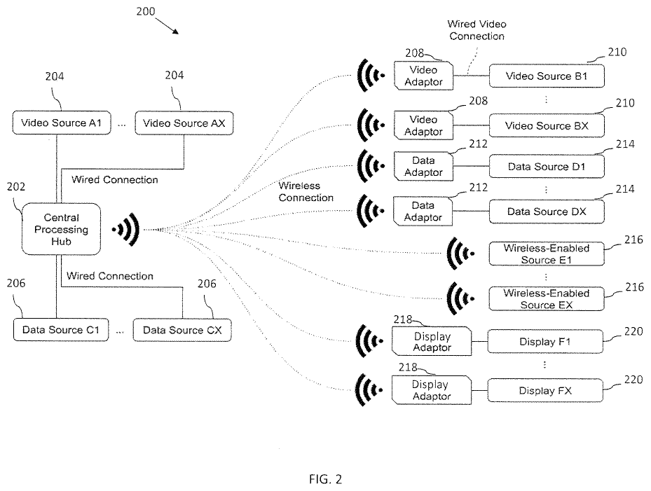

FIG. 2 depicts a system 200 using multiple media communication adaptors for wireless communication in a surgical environment according to one or more aspects. The system 200 can include a central processing hub 202 that can communicate with various media communication adaptors, or dongles, using wireless communication and with other sources useing wired connections, such as one or more video sources 204 and one or more data sources 206 coupled to the central processing hub 202 through wired connections. The system 200 can also include one or more video adaptors 208 configured to perform localized processing of video associated with surgical data and provide the processed video and/or output of video processing to the central processing hub 202 through wireless communication.

Each of the video adaptors 208 can be paired with at least one video source 210. For example, a wired video connection can link each video source 210 to a video adaptor 208. The wired connection can be a direct socket/plug connection, where the video adaptor 208 plugs into a video port or data port of the video source 210, or a wire/cable can separate the video source 210 and video adaptor 208. The video source 210 can be an output of a digital camera or other output, such as an output user interface that may be presented on a display device. The wired video connection can use any type of video transmission format supported by both the video source 210 and video adaptor 208. The localized processing of video at the video adaptors 208 can include format conversions such that video sources 210 having different video formats (e.g., video standards, video frequency, video resolution, and/or other such characteristics) may be converted and normalized to a same format before transmission to the central processing hub 202. This conversion can reduce processing burdens at the central processing hub 202 and make interpretation and analysis of multiple video streams faster. Further, the video adaptors 208 can be configured to perform one or more detection processes and provide detection information through wireless communication to the central processing hub 202 to support making one or more predictions (e.g., surgical phase predictions). As another example, the video adaptors 208 can perform one or more identification processes and provide identification information through wireless communication to the central processing hub 202 to support making the one or more predictions (e.g., surgical phase predictions).

The system 200 can also include one or more data adaptors 212 configured to perform localized processing of surgical data associated with a surgical procedure and provide the surgical data to the central processing hub 202 through wireless communication. For example, a wired data connection can link each data source 214 to a data adaptor 212. The wired connection can be a direct socket/plug connection, where the data adaptor 212 plugs into a data port or network port of the data source 214, or a wire/cable can separate the data source 214 and data adaptor 212. The data source 214 can be an output of a surgical instrument. The processing performed by the data adaptors 212 can include format conversions. For example, various surgical instruments may output data using different data formats. The data adaptors 212 can be configured to form data packets in a consistent format for consumption by the central processing hub 202. For instance, raw sensor type data may be annotated with metadata to identify the data source 214, include timestamp information, perform unit conversions, and other such modifications. Similar to the video-based example, the data adaptors 212 can be configured to perform one or more detection processes and provide detection information through wireless communication to the central processing hub 202 to support making the one or more predictions (e.g., surgical phase predictions). As another example, the data adaptors 212 can perform one or more identification processes and provide identification information through wireless communication to the central processing hub 202 to support making the one or more predictions (e.g., surgical phase predictions).

The central processing hub 202 can also receive one or more wireless communications from one or more wireless-enabled sources 216. The wireless-enabled sources 216 can support sending/receiving data or video from sources that already have wireless communication capability with the central processing hub 202. In some examples, one or more display adaptors 218 can be configured to establish wireless communication with the central processing hub 202 and output display data to one or more display devices 220. The display adaptors 218 can enable the central processing hub 202 to generate user interfaces on the display devices 220. The display devices 220 can be any type of video display or computer monitor, for example. The central processing hub 202 may customize the output sent to a display adaptor 218 based on identifying a user (e.g., actor 112 of FIG. 1) at the display device 220 using login credentials or through identifying the user through video from one of the video sources 210 as sent by one of the video adaptors 208. Facial recognition may be performed locally at one of the video adaptors 208 to identify a particular user from a group of profiles of registered users. In some examples, the display device 220 may have an integrated camera, and the display adapter 218 can identify the user based on facial recognition. An aspect of a user interface to display on the display device 220 can be customized by the display adapter 218, for example, based on identifying the user.

Thus, the system 200 of FIG. 2 can implement portions of the CAS system 100 of FIG. 1 in a distributed architecture. For instance, portions of the computing system 102 can be implemented by the central processing hub 202. Portions of the processing performed by the computing system 102 can be pushed to the video adaptors 208, data adaptors 212, and/or display adaptors 218 as edge nodes to share the computational burden. The video recording system 104 can be distributed between the central processing hub 202, the video adaptors 208, and/or the display adaptors 218. The surgical instrumentation system 106 can be distributed between the central processing hub 202 and the data adaptors 212. The data collection system 150 can be formed through a combination of the video sources 204 and data sources 206.

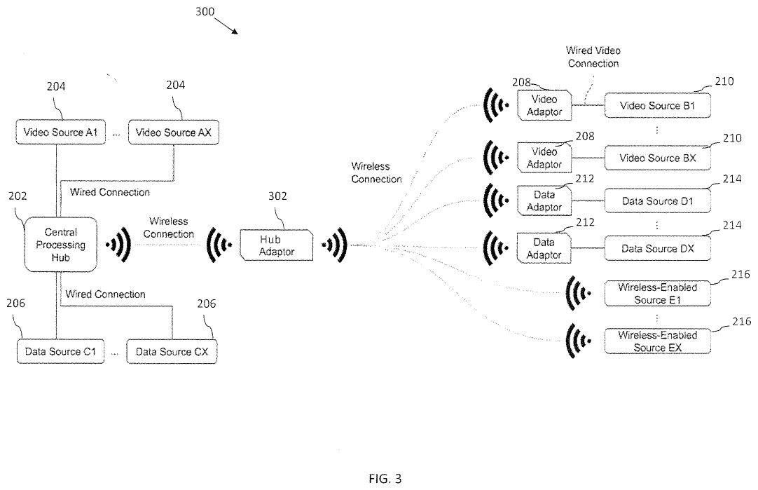

FIG. 3 depicts a system 300 using a hub adaptor 302 and multiple media communication adaptors for wireless communication in a surgical environment according to one or more aspects. The system 300 of FIG. 3 includes components as previously described with respect to FIG. 2, such as central processing hub 202, one or more video sources 204, one or more data sources 206, one or more video adaptors 208, one or more video sources 210, one or more data adaptors 212, one or more data source 214, and one or more wireless-enabled sources 216. The hub adaptor 302 (also referred to as a local hub adaptor) can be coupled by a wireless connection to the central processing hub 202 and through two or more wireless connections to the one or more data adaptors 212 and the one or more one or more video adaptors 208. Further, the hub adaptor 302 can be wirelessly coupled to the one or more wireless-enabled sources 216. Although only one hub adaptor 302 is depicted, there can be multiple hub adaptors 302 groups with various sources. For example, there can be separate hub adaptors 302 in different operating rooms and each hub adaptor 302 can establish a wireless connection with the central processing hub 202. The hub adaptor 302 can act as a localized hub for preprocessing transmissions received from an associated group of adaptors 208, 212, and/or wireless-enabled sources 216. The preprocessing can include data/video filtering, grouping, sequencing, format conversions, machine learning, localized artificial intelligence processing, and/or other such actions. The hub adaptor 302 can also serve as a security gateway or firewall to limit access and protect potentially sensitive data/video that may be accessible through the adaptors 208, 212, and/or wireless-enabled sources 216.

In some examples, the hub adaptor 302 can use a different wireless

communication protocol for transmission to/from the adaptors 208, 212, and/or wireless-enabled sources 216 as compared to transmissions to/from the central processing hub 202. The use of different communication protocols may reduce the risk of the central processing hub 202 directly connecting with a portion of the adaptors 208, 212, and/or wireless-enabled sources 216 and bypassing the formatting and security features added by the hub adaptor 302. The hub adaptor 302 may also serve as a signal power booster such that a wireless broadcast range can be extended with respect to the physical location of the central processing hub 202. Further, multiple instances of the hub adaptor 302 may be able to communicate with each other, for instance, to form a mesh network. Where one of the adaptors 208, 212, and/or wireless-enabled sources 216 is physically movable, the associated adaptors 208, 212, and/or wireless-enabled sources 216 may be able to switch broadcast groups to attach to a different hub adaptor 302 based on wireless signal quality and other such factors. Similar to the example of FIG. 2, the system 300 can be a distributed version of the CAS system 100 of FIG. I that adds further communication options through the hub adaptor 302. Further, the hub adaptor 302 can establish one or more wireless connections to one or more wireless display devices that can present information from the central processing hub 202 and/or information resulting from local processing of the hub adaptor 302.

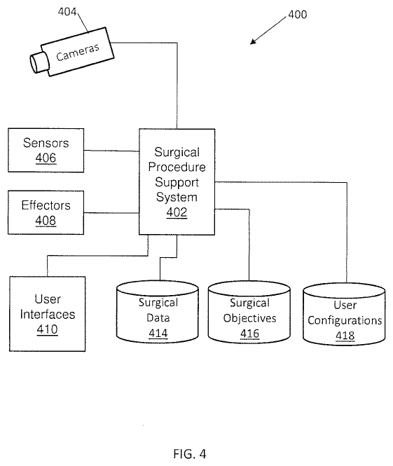

FIG. 4 depicts a surgical procedure system 400 in accordance with one or more aspects. The example of FIG. 4 depicts a surgical procedure support system 402 that can include or may be coupled to the system 100 of FIG. 1. The surgical procedure support system 402 can acquire image or video data using one or more cameras 404, such as cameras 105 of FIG. 1. The surgical procedure support system 402 can also interface with a plurality of sensors 406 and effectors 408. The sensors 406 may be associated with surgical support equipment and/or patient monitoring. The effectors 408 can be robotic components or other equipment controllable through the surgical procedure support system 402. The surgical procedure support system 402 can also interact with one or more user interfaces 410, such as various input and/or output devices. The surgical procedure support system 402 can store, access, and/or update surgical data 414 associated with a training dataset and/or live data as a surgical procedure is being performed on patient 110 of FIG. 1. The surgical procedure support system 402 can store, access, and/or update surgical objectives 416 to assist in training and guidance for one or more surgical procedures. User configurations 418 can track and store user preferences.

When implemented using the configuration of system 200 of FIG. 2, for example, the cameras 404 are an example of the video sources 210 that may be wirelessly linked through video adaptors 208 to the surgical procedure support system 402. The surgical procedure support system 402 can be formed in part by the central processing hub 202. Sensors 406 are an example of data sources 214 that can be wirelessly linked through data adaptors 212 to the surgical procedure support system 402. Effectors 408 are another example of devices that can be wirelessly linked through an adaptor, such as one of the data adaptors 212. The user interfaces 410 can be displayed on one or more of the display device 220 and can be customized according to the user configurations 418. The surgical data 414 and surgical objectives 416 can be stored, for example, as part of the video sources 204 and/or data sources 206.

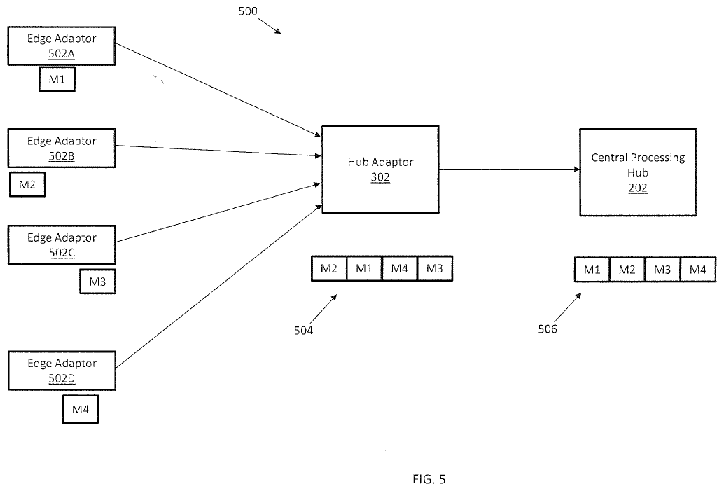

FIG. 5 depicts a block diagram 500 of adaptor synchronization in a surgical environment according to one or more aspects. In the example of FIG. 5, a hub adaptor 302 is configured to wirelessly communicate with central processing hub 202 as previously described with respect to FIG. 3. In this example, four edge adaptors 502A, 502B, 502C, 502D are configured to wirelessly communicate with the hub adaptor 302. The edge adaptors 502A-502D can be any type of adaptor or device configured to transmit wireless data to the hub adaptor 302. For example, the edge adaptors 502A-502D may be a group of related video adaptors 208 or data adaptors 212. Although separately depicted, in some aspects, one of the edge adaptors 502A-502D may function as the hub adaptor 302, thereby eliminating a separate system component.

Depending on factors, such as clock frequency differences, clock drift, communication errors, and other such conditions, messages from the edge adaptors 502A-502D, which were expected to be received in a particular sequence or synchronously, may be misaligned with respect to time. For instance, an expected message sequence may be message M1 from edge adaptor 502A, followed by message M2 from edge adaptor 502B, followed by message M3 from edge adaptor 502C, followed by message M4 from edge adaptor 502D. However, a message sequence 504 as received at the hub adaptor 302 may be messages M2, M1, M4, M3. The hub adaptor 302 can be configured to synchronize the messages and output a consistent expected pattern as output sequence 506 with messages M1, M2, M3, M4 in the expected order. The hub adaptor 302 may monitor the received sequence of messages and adjust the order of the output sequence 506 as needed. Thus, if in a subsequent transmission group, the hub adaptor 302 receives the messages as M4, M1, M3, M2, the transmission sent to the central processing hub 202 can be adjusted such that the output sequence 506 with messages M1, M2, M3, M4 is maintained in the expected order. Other variations of synchronization and order adjustment are contemplated. For example, surgical data from one or more data adaptors 212 of FIG. 2 can be synchronized with the video from one or more video adaptors 208 of FIG. 2 by the hub adaptor 302.

As a further example of synchronization adjustments, one or more of the video adaptors 208 may stream video from different video sources 210 to the hub adaptor 302.

The video sources 210 may be unsynchronized as the video adaptors 208 can operate with independent clock sources. The hub adaptor 302 can generate a synchronized video stream that aligns video streams from the video sources 210 using a common clock. The synchronized video stream can be sent to the central processing hub 202, a display adaptor 218, and/or another adaptor for further processing or display. The alignment to a common clock can reduce processing burdens for subsequent processing that operates on the video streams collectively.

As an example, each of the edge adaptors 502A-502D can be associated with a robotic surgery arm or tool, where synchronization provided by the hub adaptor 302 can assist with maintaining coordination. Further, synchronization can include aligning two or more asynchronous inputs that may operate at different data rates. For instance, two robotic arms may have sensors that generate data at a first rate, and a video stream can be received at a second rate. The hub adaptor 302 can be configured to convert the asynchronous streams into a synchronized stream for wireless transmission to the central processing hub 202. Other data sources, such as electronic medical records and/or patient scheduling information can be incorporated in synchronization data, for instance, to support further offline processing. For example, the hub adaptor 302 can tag data or video streams with identifiers that link to electronic medical records or scheduling information such that the surgical procedure information is traceable, if needed, without directly including patient information within the processed data and/or video.

Additionally, exemplary aspects of technical solutions described herein relate to, among other things, devices, systems, methods, computer-readable media, techniques, and methodologies for using machine learning and computer vision to automatically predict or detect surgical phases and instruments in surgical data. More generally, aspects can include detection, tracking, and predictions associated with one or more structures, the structures being deemed to be critical for an actor involved in performing one or more actions during a surgical procedure (e.g., by a surgeon). Prediction, tracking and/or detection processing can be distributed between multiple processing components, such as the central processing hub 202, hub adaptor 302, and various adaptors (e.g., adaptors 208, 212, 218, 502). In one or more aspects, the structures are predicted dynamically and substantially in real-time as the surgical data, including the video, is being captured and analyzed by technical solutions described herein. A predicted structure can be an anatomical structure, a surgical instrument, etc.

The surgical data provided to train the machine learning models can include data captured during a surgical procedure and simulated data. The surgical data can include time-varying image data (e.g., a simulated/real video stream from different types of cameras) corresponding to a surgical environment. The surgical data can also include other types of data streams, such as audio, radio frequency identifier (RFID), text, robotic sensors, other signals, etc. The machine learning models are trained to predict and identify, in the surgical data, “structures,” including particular tools, anatomic objects, actions being performed in the simulated/real surgical stages. In one or more aspects, the machine learning models are trained to define one or more models' parameters to learn how to transform new input data (that the models are not trained on) to identify one or more structures. During the training, the models receive, as input, one or more data streams that may be augmented with data indicating the structures in the data streams, such as indicated by metadata and/or image-segmentation data associated with the input data. The data used during training can also include temporal sequences of one or more input data.

In one or more aspects, the simulated data can be generated to include image data (e.g., which can include time-series image data or video data and can be generated in any wavelength of sensitivity) that is associated with variable perspectives, camera poses, lighting (e.g., intensity, hue, etc.) and/or motion of imaged objects (e.g., tools). In some instances, multiple data sets can be generated-each of which corresponds to the same imaged virtual scene but varies with respect to perspective, camera pose, lighting, and/or motion of imaged objects, or varies with respect to the modality used for sensing, e.g., red-green-blue (RGB) images or depth or temperature. In some instances, each of the multiple data sets corresponds to a different imaged virtual scene and further varies with respect to perspective, camera pose, lighting, and/or motion of imaged objects.

The machine learning models can include, for instance, a fully convolutional network adaptation (FCN) and/or conditional generative adversarial network model configured with one or more hyperparameters for phase and/or surgical instrument detection. For example, the machine learning models (e.g., the fully convolutional network adaptation) can be configured to perform supervised, self-supervised, or semi-supervised semantic segmentation in multiple classes-each of which corresponding to a particular surgical instrument, anatomical body part (e.g., generally or in a particular state), and/or environment. Alternatively, or in addition, the machine learning model (e.g., the conditional generative adversarial network model) can be configured to perform unsupervised domain adaptation to translate simulated images to semantic instrument segmentations. It is understood that other types of machine learning models or combinations thereof can be used in one or more aspects. Machine learning models can further be trained to perform surgical phase detection and may be developed for a variety of surgical workflows, as further described herein. Machine learning models can be collectively managed as a group, also referred to as an ensemble, where the machine learning models are used together and may share feature spaces between elements of the models. As such, reference to a machine learning model or machine learning models herein may refer to a combination of multiple machine learning models that are used together, such as operating on the same group of data. Although specific examples are described with respect to types of machine learning models, other machine learning and/or deep learning techniques can be used to implement the features described herein.

In one or more aspects, one or more machine learning models are trained using a joint training process to find correlations between multiple tasks that can be observed and predicted based on a shared set of input data. Further machine learning refinements can be achieved by using a portion of a previously trained machine learning network to further label or refine a training dataset used in training the one or more machine learning models. For example, semi-supervised or self-supervised learning can be used to initially train the one or more machine learning models using partially annotated input data as a training dataset. The partially annotated training dataset may be missing labels on some of the data associated with a particular input, such as missing labels on instrument data.

An instrument network learned as part of the one or more machine learning models can be applied to the partially annotated training dataset to add missing labels to partially labeled instrument data in the training dataset. The updated training dataset with at least a portion of the missing labels populated can be used to further train the one or more machine learning models. This iterative training process may result in model size compression for faster performance and can improve overall accuracy by training ensembles. Ensemble performance improvement can result where feature sets are shared such that feature sets related to surgical instruments are also used for surgical phase detection, for example. Thus, improving the performance aspects of machine learning related to instrument data may also improve the performance of other networks that are primarily directed to other tasks.

After training, the one or more machine learning models can then be used in real-time to process one or more data streams (e.g., video streams, audio streams, RFID data, etc.). The processing can include predicting and characterizing one or more surgical phases, instruments, and/or other structures within various instantaneous or block time periods. The results can then be used to identify the presence, localization, and/or use of one or more features. For example, the localization of surgical instrument(s) can include a bounding box, a medial axis, and/or any other marker or key point identifying the location of the surgical instrument(s). Various approaches to localization can be performed individually or jointly. The localization can be represented as coordinates in images that map to pixels depicting the surgical instrument(s) in the images. Localization of other structures, such as anatomical structures, can be used to provide locations, e.g., coordinates, heatmaps, bounding boxes, boundaries, masks, etc., of one or more anatomical structures identified and distinguish between other structures, such as surgical instruments. Anatomical structures can include organs, arteries, implants, surgical artifacts (e.g., staples, stitches, etc.), etc. A “location” of a detected feature, such as an anatomical structure, surgical instrument, etc., can be specified as multiple sets of coordinates (e.g., polygon), a single set of coordinates (e.g., centroid), or any other such manner without limiting the technical features described herein.

Alternatively, or in addition, the structures can be used to identify a stage within a surgical workflow (e.g., as represented via a surgical data structure), predict a future stage within a workflow, the remaining time of the operation, etc. Workflows can be segmented into a hierarchy, such as events, actions, steps, surgical objectives, phases, complications, and deviations from a standard workflow. For example, an event can be camera in, camera out, bleeding, leak test, etc. Actions can include surgical activities being performed, such as incision, grasping, etc. Steps can include lower-level tasks as part of performing an action, such as first stapler firing, second stapler firing, etc. Surgical objectives can define a desired outcome during surgery, such as gastric sleeve creation, gastric pouch creation, etc. Phases can define a state during a surgical procedure, such as preparation, surgery, closure, etc. Complications can define problems, or abnormal situations, such as hemorrhaging, staple dislodging, etc. Deviations can include alternative routes indicative of any type of change from a previously learned workflow. Aspects can include workflow detection and prediction, as further described herein.

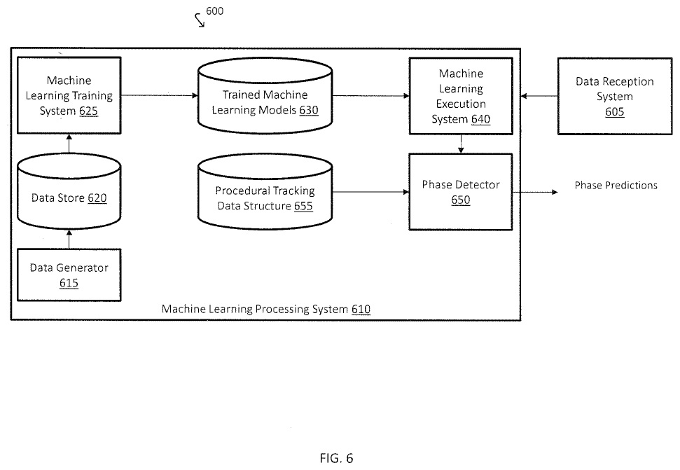

FIG. 6 shows a system 600 for analyzing video and data according to one or more aspects. For example, the video and data can be captured from video sources 210 and data sources 214 of FIG. 2. Portions of the surgical machine learning process can be performed in part on the video adaptors 208 and/or data adaptors 212 as localized processing of the surgical data. The central processing hub 202 of FIG. 2 can incorporate local processing results of the video adaptors 208 and/or data adaptors 212 in combination with video sources 204 and/or data sources 206 in a surgical machine learning process. The analysis can result in predicting surgical phases and structures (e.g., instruments, anatomical structures, etc.) in the video data using machine learning. The system 600 can be the computing system 102 of FIG. 1, or a part thereof in one or more examples. System 600 uses data streams in the surgical data to identify procedural states according to some aspects.

System 600 includes a data reception system 605 that collects surgical data, including the video data and surgical instrumentation data. The data reception system 605 can include one or more devices (e.g., one or more user devices and/or servers) located within and/or associated with a surgical operating room and/or control center. The data reception system 605 can receive surgical data in real-time, i.e., as the surgical procedure is being performed. Alternatively, or in addition, the data reception system 605 can receive or access surgical data in an offline manner, for example, by accessing data that is stored in the data collection system 150 of FIG. 1.

System 600 further includes a machine learning processing system 610 that processes the surgical data using one or more machine learning models to identify one or more features, such as surgical phase, instrument, anatomical structure, etc., in the surgical data. It will be appreciated that machine learning processing system 610 can include one or more devices (e.g., one or more servers), each of which can be configured to include part or all of one or more of the depicted components of the machine learning processing system 610. In some instances, a part or all of the machine learning processing system 610 is in the cloud and/or remote from an operating room and/or physical location corresponding to a part or all of data reception system 605. It will be appreciated that several components of the machine learning processing system 610 are depicted and described herein. However, the components are just one example structure of the machine learning processing system 610, and that in other examples, the machine learning processing system 610 can be structured using a different combination of the components. Such variations in the combination of the components are encompassed by the technical solutions described herein.

The machine learning processing system 610 includes a machine learning training system 625, which can be a separate device (e.g., server) that stores its output as one or more trained machine learning models 630. The machine learning models 630 are accessible by a model execution system 640. The model execution system 640 can be separate from the machine learning training system 625 in some examples. In other words, in some aspects, devices that “train” the models are separate from devices that “infer,” i.e., perform real-time processing of surgical data using the trained machine learning models 630.

Machine learning processing system 610, in some examples, further includes a data generator 615 to generate simulated surgical data, such as a set of virtual images, or record the video data from the video recording system 104, to train the machine learning models 630. Data generator 615 can access (read/write) a data store 620 to record data, including multiple images and/or multiple videos. The images and/or videos can include images and/or videos collected during one or more procedures (e.g., one or more surgical procedures). For example, the images and/or video may have been collected by a user device worn by the actor 112 of FIG. 1 (e.g., surgeon, surgical nurse, anesthesiologist, etc.) during the surgery, a non-wearable imaging device located within an operating room, or an endoscopic camera inserted inside the patient 110 of FIG. 1. The data store 620 is separate from the data collection system 150 of FIG. 1 in some examples. In other examples, the data store 620 is part of the data collection system 150.

Each of the images and/or videos recorded in the data store 620 for training the machine learning models 630 can be defined as a base image and can be associated with other data that characterizes an associated procedure and/or rendering specifications. For example, the other data can identify a type of procedure, a location of a procedure, one or more people involved in performing the procedure, surgical objectives, and/or an outcome of the procedure. Alternatively, or in addition, the other data can indicate a stage of the procedure with which the image or video corresponds, rendering specification with which the image or video corresponds and/or a type of imaging device that captured the image or video (e.g., and/or, if the device is a wearable device, a role of a particular person wearing the device, etc.). Further, the other data can include image-segmentation data that identifies and/or characterizes one or more objects (e.g., tools, anatomical objects, etc.) that are depicted in the image or video. The characterization can indicate the position, orientation, or pose of the object in the image. For example, the characterization can indicate a set of pixels that correspond to the object and/or a state of the object resulting from a past or current user handling. Localization can be performed using a variety of techniques for identifying objects in one or more coordinate systems.

The machine learning training system 625 uses the recorded data in the data

store 620, which can include the simulated surgical data (e.g., set of virtual images) and actual surgical data to train the machine learning models 630. The machine learning model 630 can be defined based on a type of model and a set of hyperparameters (e.g., defined based on input from a client device). The machine learning models 630 can be configured based on a set of parameters that can be dynamically defined based on (e.g., continuous or repeated) training (i.e., learning, parameter tuning). Machine learning training system 625 can use one or more optimization algorithms to define the set of parameters to minimize or maximize one or more loss functions. The set of (learned) parameters can be stored as part of a trained machine learning model 630 using a specific data structure for that trained machine learning model 630. The data structure can also include one or more non-learnable variables (e.g., hyperparameters and/or model definitions).

Machine learning execution system 640 can access the data structure(s) of the machine learning models 630 and accordingly configure the machine learning models 630 for inference (i.e., prediction). The machine learning models 630 can include, for example, a fully convolutional network adaptation, an adversarial network model, an encoder, a decoder, or other types of machine learning models. The type of the machine learning models 630 can be indicated in the corresponding data structures. The machine learning model 630 can be configured in accordance with one or more hyperparameters and the set of learned parameters.

The machine learning models 630, during execution, receive, as input, surgical data to be processed and subsequently generate one or more inferences according to the training. For example, the video data captured by the video recording system 104 of FIG. 1 can include data streams (e.g., an array of intensity, depth, and/or RGB values) for a single image or for each of a set of frames (e.g., including multiple images or an image with sequencing data) representing a temporal window of fixed or variable length in a video. The video data that is captured by the video recording system 104 can be received by the data reception system 605, which can include one or more devices located within an operating room where the surgical procedure is being performed. Alternatively, the data reception system 605 can include devices that are located remotely, to which the captured video data is streamed live during the performance of the surgical procedure. Alternatively, or in addition, the data reception system 605 accesses the data in an offline manner from the data collection system 150 or from any other data source (e.g., local or remote storage device).

The data reception system 605 can process the video and/or data received. The processing can include decoding when a video stream is received in an encoded format such that data for a sequence of images can be extracted and processed. The data reception system 605 can also process other types of data included in the input surgical data. For example, the surgical data can include additional data streams, such as audio data, RFID data, textual data, measurements from one or more surgical instruments/sensors, etc., that can represent stimuli/procedural states from the operating room. The data reception system 605 synchronizes the different inputs from the different devices/sensors before inputting them in the machine learning processing system 610.

The machine learning models 630, once trained, can analyze the input surgical data, and in one or more aspects, predict and/or characterize structures included in the video data included with the surgical data. The video data can include sequential images and/or encoded video data (e.g., using digital video file/stream formats and/or codecs, such as MP4, MOV, AVI, WEBM, AVCHD, OGG, etc.). The prediction and/or characterization of the structures can include segmenting the video data or predicting the localization of the structures with a probabilistic heatmap. In some instances, the one or more machine learning models include or are associated with a preprocessing or augmentation (e.g., intensity normalization, resizing, cropping, etc.) that is performed prior to segmenting the video data. An output of the one or more machine learning models can include image-segmentation or probabilistic heatmap data that indicates which (if any) of a defined set of structures are predicted within the video data, a location and/or position and/or pose of the structure(s) within the video data, and/or state of the structure(s). The location can be a set of coordinates in an image/frame in the video data. For example, the coordinates can provide a bounding box. The coordinates can provide boundaries that surround the structure(s) being predicted. The machine learning models 630, in one or more examples, are trained to perform higher-level predictions and tracking, such as predicting a phase of a surgical procedure and tracking one or more surgical instruments used in the surgical procedure.

While some techniques for predicting a surgical phase (“phase”) in the surgical procedure are described herein, it should be understood that any other technique for phase prediction can be used without affecting the aspects of the technical solutions described herein. In some examples, the machine learning processing system 610 includes a phase detector 650 that uses the machine learning models to identify a phase within the surgical procedure (“procedure”). Phase detector 650 uses a particular procedural tracking data structure 655 from a list of procedural tracking data structures. Phase detector 650 selects the procedural tracking data structure 655 based on the type of surgical procedure that is being performed. In one or more examples, the type of surgical procedure is predetermined or input by actor 112. The procedural tracking data structure 655 identifies a set of potential phases that can correspond to a part of the specific type of procedure.

In some examples, the procedural tracking data structure 655 can be a graph that includes a set of nodes and a set of edges, with each node corresponding to a potential phase. The edges can provide directional connections between nodes that indicate (via the direction) an expected order during which the phases will be encountered throughout an iteration of the procedure. The procedural tracking data structure 655 may include one or more branching nodes that feed to multiple next nodes and/or can include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state can indicate a complication (e.g., blood clots, clogged arteries/veins, etc.), pre-condition (e.g., lesions, polyps, etc.). In some examples, the machine learning models 630 are trained to detect an “abnormal condition,” such as hemorrhaging, arrhythmias, blood vessel abnormality, etc.

Each node within the procedural tracking data structure 655 can identify one or more characteristics of the phase corresponding to that node. The characteristics can include visual characteristics. In some instances, the node identifies one or more tools that are typically in use or availed for use (e.g., on a tool tray) during the phase. The node also identifies one or more roles of people who are typically performing a surgical task, a typical type of movement (e.g., of a hand or tool), etc. Thus, phase detector 650 can use the segmented data generated by model execution system 640 that indicates the presence and/or characteristics of particular objects within a field of view to identify an estimated node to which the real image data corresponds. Identification of the node (i.e., phase) can further be based upon previously detected phases for a given procedural iteration and/or other detected input (e.g., verbal audio data that includes person-to-person requests or comments, explicit identifications of a current or past phase, information requests, etc.).

The phase detector 650 outputs the phase prediction associated with a portion of the video data that is analyzed by the machine learning processing system 610. The phase prediction is associated with the portion of the video data by identifying a start time and an end time of the portion of the video that is analyzed by the machine learning execution system 640. The phase prediction that is output can include an identity of a surgical phase as detected by the phase detector 650 based on the output of the machine learning execution system 640. Further, the phase prediction, in one or more examples, can include identities of the structures (e.g., instrument, anatomy, etc.) that are identified by the machine learning execution system 640 in the portion of the video that is analyzed. The phase prediction can also include a confidence score of the prediction. Other examples can include various other types of information in the phase prediction that is output.

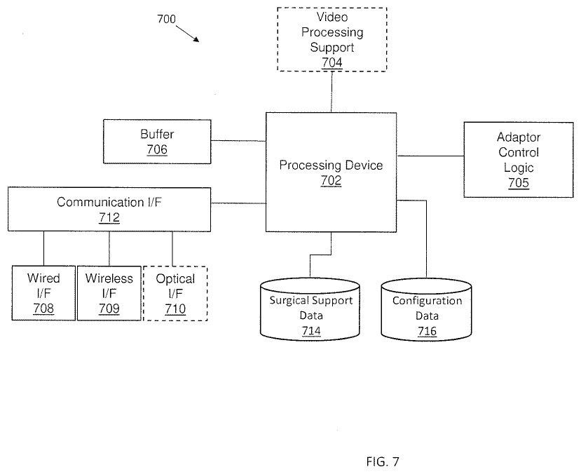

FIG. 7 depicts a block diagram of an adaptor 700 for a surgical environment according to one or more aspects. The adaptor 700 is depicted as a generalized adaptor or dongle that can be used to implement one or more of the previously described adaptors, such as video adaptor 208, data adaptor 212, display adaptor 218, hub adaptor 302, and/or edge adaptor 502A-502D. Further, the central processing hub 202 can be one of the adaptors 700. The adaptor 700 can include at least one processing device 702 configured to execute instructions and/or implement circuits. For instance, the processing device 702 can be a microcontroller, a field programmable gate array, an application specific integrated circuit, a digital signal processor, or other such device capable of executing instructions. In some aspects, the adaptor 700 can include video processing support 704, such as a graphics card or graphics processing unit. Video processing support 704 can support video capture in various video formats. The graphics processing unit of the video processing support 704 may also or alternatively be used to support machine learning process execution locally on the adaptor 700.

The adaptor 700 can include a buffer 706 for temporary storage of video and/or data for preprocessing before forwarding the processed result. For example, data or video can be received through a wired interface 708 or an optical interface 710 of a communication interface 712 and stored in the buffer 706. The wired interface 708 may support various digital and/or analog connections, such as connections using coaxial cables, HDMI cables, Ethernet cables, USB connectors, and the like. Adaptor control logic 705 can include circuitry or instructions executable by the processing device 702 to determine what type of processing to perform on the buffered data/video. Where surgery data processing is supported, surgical support data 714 can be accessed to assist in determining various aspects of surgical phase predictions, detection, identification, tracking, and/or other processing, for instance, as part of implementing the system 600 of FIG. 6. Upon performing processing, conversion, and/or synchronization operations, the resulting data/video/messages can be transmitted through a wireless interface 709 coupled to the communication interface 712. The communication interface 712 can support intelligent connect and discovery of wireless connections through the wireless interface 709. The communication interface 712 may also include supplemental wireless technology to support wireless identification and discovery separate from the wireless communications performed through the wireless interface 709. Near-field communication, Bluetooth, ultra-wideband chip technology and other technologies may be used for this purpose. The wireless interface 709 may include a Wi-Fi chip that supports specific radio frequency transmissions with various security features.

The adaptor 700 can support multiple pairing and discovery options with other devices, such as pairing with other adaptors 700, the central processing hub 202, video sources 210, data sources 214, wireless-enabled sources 216, displays 220, and the like. Pairing can be supported, for example, in an automatic, semi-automatic, or manual pairing mode. For instance, pairing modes can use one or more authentication techniques through passwords, quick response codes, infrared communication, Bluetooth, radio frequency identification, near-field communication, audio-based communication, and other such approaches to establish communication links. Adaptive pairing of devices can support flexible configuration and reconfiguration of the communication network as devices are added or removed. Automatic pairing may require no human user intervention, while semi-automatic pairing may attempt to connect upon a user authorization or consent. Manual pairing can include direct user interactions with one or more devices to establish communication.

The adaptor 700 can also store configuration data 716 to define, for example,

whether the adaptor 700 is configured to perform as a video adaptor 208, data adaptor 212, display adaptor 218, hub adaptor 302, edge adaptor 502A-502D and/or central processing hub 202. The adaptor 700 can support reconfiguration of various parameters. For example, the central processing hub 202 can send one or more configuration commands through wireless communication to reconfigure operation of the adaptor 700.

For instance, when implemented as a data adaptor 212 or video adaptor 208, the adaptor 700 can be reconfigured to change communication protocols, conversion protocols, connection support parameters, preprocessing operations, security, and/or other such aspects. Once the adaptor 700 is powered or reset, the adaptor 700 can start streaming data/video without requiring additional external commands.

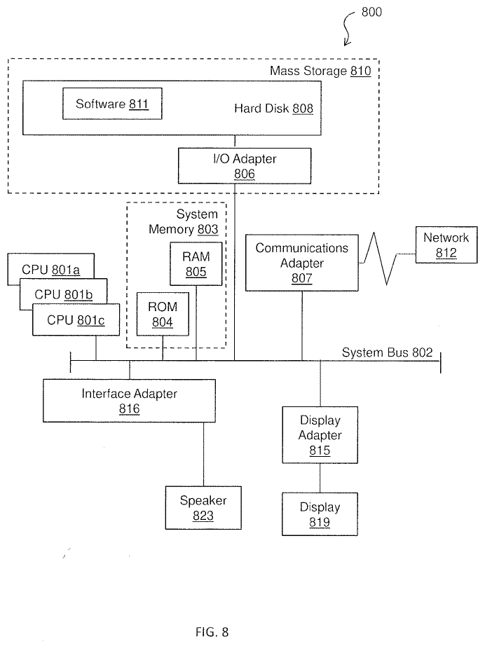

Turning now to FIG. 8, a computer system 800 is generally shown in accordance with an aspect. The computer system 800 can be an electronic computer framework comprising and/or employing any number and combination of computing devices and networks utilizing various communication technologies, as described herein.

The computer system 800 can be easily scalable, extensible, and modular, with the ability to change to different services or reconfigure some features independently of others. The computer system 800 may be, for example, a server, desktop computer, laptop computer, tablet computer, or smartphone. In some examples, computer system 800 may be a cloud computing node. Computer system 800 may be described in the general context of computer-executable instructions, such as program modules, being executed by a computer system. Generally, program modules may include routines, programs, objects, components, logic, data structures, and so on that perform particular tasks or implement particular abstract data types. Computer system 800 may be practiced in distributed cloud computing environments where tasks are performed by remote processing devices that are linked through a communications network. In a distributed cloud computing environment, program modules may be located in both local and remote computer system storage media, including memory storage devices.

As shown in FIG. 8, the computer system 800 has one or more central processing units (CPU(s)) 801a, 801b, 801c, etc. (collectively or generically referred to as processor(s) 801). The processors 801 can be a single-core processor, multi-core processor, computing cluster, or any number of other configurations. The processors 801, also referred to as processing circuits, are coupled via a system bus 802 to a system memory 803 and various other components. The system memory 803 can include one or more memory devices, such as read-only memory (ROM) 804 and a random access memory (RAM) 805. The ROM 804 is coupled to the system bus 802 and may include a basic input/output system (BIOS), which controls certain basic functions of the computer system 800. The RAM is read-write memory coupled to the system bus 802 for use by the processors 801. The system memory 803 provides temporary memory space for operations of said instructions during operation. The system memory 803 can include random access memory (RAM), read-only memory, flash memory, or any other suitable memory systems.

The computer system 800 comprises an input/output (I/O) adapter 806 and a communications adapter 807 coupled to the system bus 802. The I/O adapter 806 may be a small computer system interface (SCSI) adapter that communicates with a hard disk 808 and/or any other similar component. The I/O adapter 806 and the hard disk 808 are collectively referred to herein as a mass storage 810.

Software 811 for execution on the computer system 800 may be stored in the mass storage 810. The mass storage 810 is an example of a tangible storage medium readable by the processors 801, where the software 811 is stored as instructions for execution by the processors 801 to cause the computer system 800 to operate, such as is described hereinbelow with respect to the various Figures. Examples of computer program product and the execution of such instruction is discussed herein in more detail. The communications adapter 807 interconnects the system bus 802 with a network 812, which may be an outside network, enabling the computer system 800 to communicate with other such systems. In one aspect, a portion of the system memory 803 and the mass storage 810 collectively store an operating system, which may be any appropriate operating system to coordinate the functions of the various components shown in FIG. 8.

Additional input/output devices are shown as connected to the system bus 802 via a display adapter 815 and an interface adapter 816 and. In one aspect, the adapters 806, 807, 815, and 816 may be connected to one or more I/O buses that are connected to the system bus 802 via an intermediate bus bridge (not shown). A display 819 (e.g., a screen or a display monitor) is connected to the system bus 802 by a display adapter 815, which may include a graphics controller to improve the performance of graphics-intensive applications and a video controller. A keyboard, a mouse, a touchscreen, one or more buttons, a speaker, etc., can be interconnected to the system bus 802 via the interface adapter 816, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 8, the computer system 800 includes processing capability in the form of the processors 801, and, storage capability including the system memory 803 and the mass storage 810, input means such as the buttons, touchscreen, and output capability including the speaker 823 and the display 819.

In some aspects, the communications adapter 807 can transmit data using any suitable interface or protocol, such as the internet small computer system interface, among others. The network 812 may be a cellular network, a radio network, a wide area network (WAN), a local area network (LAN), or the Internet, among others. An external computing device may connect to the computer system 800 through the network 812. In some examples, an external computing device may be an external web server or a cloud computing node.

It is to be understood that the block diagram of FIG. 8 is not intended to indicate that the computer system 800 is to include all of the components shown in FIG. 8. Rather, the computer system 800 can include any appropriate fewer or additional components not illustrated in FIG. 8 (e.g., additional memory components, embedded controllers, modules, additional network interfaces, etc.). Further, the aspects described herein with respect to computer system 800 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects.

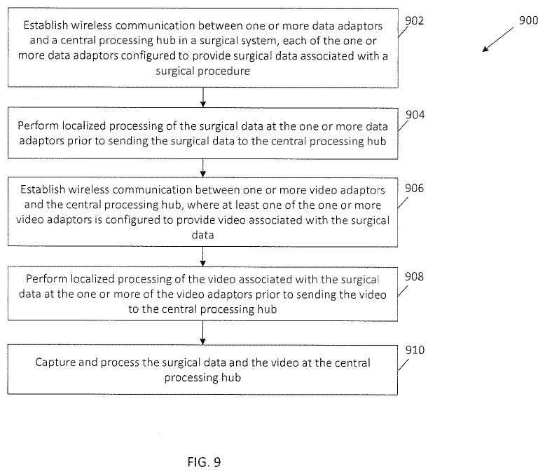

FIG. 9 depicts a flowchart of a method 900 for processing and wireless communication between multiple sources and a central processing hub in a surgical environment according to one or more aspects. Method 900 can be executed by system 200 of FIG. 2, system 300 of FIG. 3, and/or other systems as disclosed herein to perform a computer-implemented method. For purposes of explanation, the method 900 is described in reference to of FIGS. 1-9 and may be performed in an alternate order including adding, removing, combining, or subdividing steps.

At block 902, wireless communication can be established between one or more data adaptors 212 and a central processing hub 202 in a surgical system, such as system 200. Each of the one or more data adaptors 212 can be configured to provide surgical data 414 associated with a surgical procedure. At block 904, localized processing of the surgical data 414 can be performed at one or more data adaptors 212 prior to sending the surgical data 414 to the central processing hub 202. At block 906, wireless communication can be established between one or more video adaptors 208 and the central processing hub 202. At least one of the one or more video adaptors 208 can be configured to provide video associated with the surgical data 414. At block 908, localized processing of the video associated with the surgical data 414 can be performed at the one or more of the video adaptors 208 prior to sending the video to the central processing hub 202. At block 910, the surgical data 414 and the video can be captured and processed at the central processing hub 202.

In some aspects, wireless communication between the one or more data adaptors 212 and the central processing hub 202 can pass through a hub adaptor 302 configured to synchronize the surgical data 414 from the one or more data adaptors 212. Wireless communication between the one or more video adaptors 208 and the central processing hub 202 may pass through the hub adaptor 302. The hub adaptor 302 can be configured to synchronize the surgical data 414 from the one or more data adaptors 212 with the video from the one or more video adaptors 208. One of the one or more data adaptors 212 or one of the one or more video adaptors 208 can be configured to operate as the hub adaptor 302.

Video and/or data can also be received through one or more wired connections at the central processing hub 202, such as from video source 204 and/or data source 206. One or more wireless communications can be received at the central processing hub 202 from one or more wireless-enabled sources 216. Time-sensitivity networking may also be supported through the hub adaptor 302.

Wireless communication can be established between one or more display adaptors 218 and the central processing hub 202. Display data and/or video can be transmitted through one or more wireless connections to the one or more display adaptors 218 for output on one or more display devices 220 coupled to the one or more display adaptors 218. In some examples, one of the display adaptors 218 can identify a user (e.g., actor 112) of one of the display devices 220 and customize an aspect of a user interface 410 or information presented on the user interface 410 to display on one of the display devices 220 based on identifying the user.

Performing localized processing of the surgical data 414 can include performing at least a portion of a surgical machine learning process on at least one of the data adaptors 212. Further, performing localized processing of the video associated with the surgical data 414 can include modifying one or more aspects of the video based on performing at least a portion of a surgical machine learning process on at least one of the video adaptors 208. For example, the processing of method 900 can include using the machine learning processing system 610 to detect, predict, and track features, including surgical phases, anatomical structures, and instruments, in a video of a surgical procedure. Systems 100, 200, 300, 400 can process different portions of video being analyzed differently based on the phase prediction for each portion, the phase prediction output by the machine learning processing system 610. Different types of processing can include encoding different portions using a different protocol (e.g., different codecs).

As used herein, “critical anatomical structures” can be specific to the type of surgical procedure being performed and identified automatically. Additionally, a surgeon or any other user can configure the system 600 to identify particular anatomical structures as critical for a particular patient. The selected anatomical structures are critical to the success of the surgical procedure, such as anatomical landmarks (e.g., Calot triangle, Angle of His, etc.) that need to be identified during the procedure or those resulting from a previous surgical task or procedure (e.g., stapled or sutured tissue, clips, etc.). System 600 can access a plurality of surgical objectives associated with the surgical procedure and correlate the surgical objectives with the one or more surgical instruments and the phase of the surgical procedure. Observations relative to critical anatomical structures and surgical objectives can be used to control alert generation. The critical anatomical structures can be used for determining an abnormal event in some examples.

Aspects of the technical solutions described herein can improve CAS systems, particularly by facilitating data and video transfer optimizations. Further, the technical solutions described herein facilitate improvements to computing technology, particularly computing techniques used for distributed processing, storage, and transmission.

Aspects of the technical solutions described herein facilitate one or more machine learning models, such as computer vision models, to process images obtained from a live video feed of the surgical procedure in real-time using spatio-temporal information. The machine learning models using techniques such as neural networks to use information from the live video feed and (if available) robotic sensor platform to predict one or more features, such as anatomical structures, surgical instruments, in an input window of the live video feed, and further refine the predictions using additional machine learning models that can predict a phase of the surgical procedure. The machine learning models can be trained to identify the surgical phase(s) of the procedure and structures in the field of view by learning from raw image data. When in a robotic procedure, the computer vision models can also accept sensor information (e.g., instruments enabled, mounted, etc.) to improve the predictions. Computer vision models that predict instruments and critical anatomical structures use temporal information from the phase prediction models to improve the confidence of the predictions in real-time.

The predictions and the corresponding confidence scores can be used to generate and display video based on video captured during a surgical procedure. Aspects of the technical solutions described herein provide a practical application in surgical procedures and storage of large amounts of data (Terabytes, Petabytes, etc.) captured during surgical procedures.

It should be noted that although some aspects can include endoscopic video, the technical solutions described herein can be applied to analyze video and image data captured by cameras that are not endoscopic (i.e., cameras external to the patient's body) when performing open surgeries (i.e., not laparoscopic surgeries). For example, the video and image data can be captured by cameras that are mounted on one or more personnel in the operating room, e.g., surgeon. Alternatively, or in addition, the cameras can be mounted on surgical instruments, walls, or other locations in the operating room.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to aspects of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects of the present invention have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects of the invention are described herein with reference to the related drawings. Alternative aspects of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general-purpose microprocessors, application-specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A computer-implemented method comprising:
establishing wireless communication between one or more data adaptors and a central processing hub in a surgical system, each of the one or more data adaptors configured to provide surgical data associated with a surgical procedure;
performing localized processing of the surgical data at the one or more data adaptors prior to sending the surgical data to the central processing hub;
establishing wireless communication between one or more video adaptors and the central processing hub, where at least one of the one or more video adaptors is configured to provide video associated with the surgical data;
performing localized processing of the video associated with the surgical data at the one or more of the video adaptors prior to sending the video to the central processing hub; and
capturing and processing the surgical data and the video at the central processing hub.

2. The computer-implemented method of claim 1, further comprising:
receiving video and/or data through one or more wired connections at the central processing hub; and
receiving one or more wireless communications at the central processing hub from one or more wireless-enabled sources.

3. The computer-implemented method of claim 1, wherein wireless communication between the one or more data adaptors and the central processing hub passes through a hub adaptor configured to synchronize the surgical data from the one or more data adaptors and/or synchronize video received from the one or more video adaptors.

4. The computer-implemented method of claim 3, wherein wireless communication between the one or more video adaptors and the central processing hub passes through the hub adaptor, and hub adaptor is configured to synchronize the surgical data from the one or more data adaptors with the video from the one or more video adaptors.

5. The computer-implemented method of claim 4, further comprising:
configuring one of the one or more data adaptors or one of the one or more video adaptors to operate as the hub adaptor.

6. The computer-implemented method of claim 1, further comprising:
establishing wireless communication between one or more display adaptors and the central processing hub; and
transmitting display data and/or video through one or more wireless connections to the one or more display adaptors for output on one or more display devices coupled to the one or more display adaptors.

7. The computer-implemented method of claim 6, further comprising:
identifying, by one of the display adaptors, a user of one of the display devices; and
customizing an aspect of a user interface or information presented in the user interface to display on one of the display devices based on identifying the user.

8. The computer-implemented method of claim 1, wherein performing localized processing of the surgical data comprises performing at least a portion of a surgical machine learning process on at least one of the data adaptors.

9. The computer-implemented method of claim 1, wherein performing localized processing of the video associated with the surgical data comprises modifying one or more aspects of the video based on performing at least a portion of a surgical machine learning process on at least one of the video adaptors.

10. A system comprising:
a central processing hub;
one or more data adaptors configured to perform localized processing of surgical data associated with a surgical procedure and provide the surgical data to the central processing hub through wireless communication; and
one or more video adaptors configured to perform localized processing of video associated with the surgical data and provide the video to the central processing hub through wireless communication.

11. The system of claim 10, further comprising:
one or more video sources and one or more data sources coupled to the central processing hub through wired connections; and
one or more wireless-enabled sources coupled to the central processing hub.

12. The system of claim 11, further comprising:
a hub adaptor coupled by a wireless connection to the central processing hub and through two or more wireless connections to the one or more data adaptors and the one or more one or more video adaptors, wherein the hub adaptor is configured to pair with the central processing hub, the one or more data adaptors, and the one or more one or more video adaptors using a manual mode, a semi-automatic mode, or an automatic mode.

13. The system of claim 12, wherein the hub adaptor is configured to perform preprocessing of the surgical data and the video prior to sending the surgical data and the video to the central processing hub.

14. The system of claim 10, further comprising:
one or more display adaptors configured to establish wireless communication with the central processing hub and output display data to one or more display devices.

15. A computer program product comprising a memory device having computer-executable instructions stored thereon, which when executed by one or more processors cause the one or more processors to perform a method comprising:
receiving surgical data through wireless communication from one or more data adaptors at a central processing hub, wherein the one or more data adaptors are coupled to one or more data sources in a surgical system;
receiving video associated with the surgical data through wireless communication from one or more video adaptors at the central processing hub, wherein the one or more video adaptors are coupled to one or more video sources in the surgical system; and
performing a surgical machine learning process to make one or more phase predictions of a surgical procedure based on the surgical data and the video.

16. The computer program product of claim 15, wherein one or more of the data adaptors or the video adaptors perform one or more detection processes and provide detection information through wireless communication to the central processing hub to support making the one or more phase predictions.

17. The computer program product of claim 15, wherein one or more of the data adaptors or the video adaptors perform one or more identification processes and provide identification information through wireless communication to the central processing hub to support making the one or more phase predictions.

18. The computer program product of claim 17, wherein wireless communication between the one or more data adaptors and the central processing hub is modified upon passing through a hub adaptor configured to synchronize the surgical data from the one or more data adaptors and/or synchronize video received from the one or more video adaptors.

19. The computer program product of claim 15, the method further comprising:
transmitting display data through one or more wireless connections from the central processing hub to one or more display adaptors for output on one or more display devices coupled to the one or more display adaptors, wherein the display data is customized at one or more display adaptors based on identifying one or more users of the one or more display devices.

20. The computer program product of claim 15, the method further comprising:
sending one or more configuration commands through wireless communication to reconfigure operation of at least one of the data adaptors or the video adaptors to become a hub adaptor.
